(19) United States
(12) Patent Application Publication
(10) Pub. No.: US 2015/0135364 A1
(43) Pub. Date: May 14, 2015

(54) METHOD AND MATERIALS FOR MAKING PLANTS HEAT-RESISTANT

(71) Applicant: Universität Potsdam, Potsdam (DE)

(72) Inventor: Isabel Bäurle, Postdam (DE)

(21) Appl. No.: 14/534,704

(22) Filed: Nov. 6, 2014

(30) Foreign Application Priority Data
Nov. 14, 2013 (EP) 13192897.0
Jan. 22, 2014 (EP) 14152191.4

(51) Int. Cl. C12N 15/82

(52) U.S. Cl. CPC C12N 15/8271; C12N 15/8218
USPC 800/285; 435/320.1; 800/298; 435/419

(57) ABSTRACT

The invention pertains to a method for generating a heat-resistant plant, including the step of introducing a polynucleotide, having a first sequence portion for transcriptionally driving a second sequence portion that is operably linked to a second sequence portion encoding for a miRNA156 family member into a plant cell, wherein the first sequence portion is heat-stress inducible.

### CROSS-REFERENCE TO RELATED APPLICATIONS

This application claims priority to European Patent Application No. 13 192 897.0 filed Nov. 14, 2013 and European Patent Application No. 14 152 191.4 filed Jan. 21, 2014, the disclosures of which are incorporated by reference herein in their entireties.

### FIELD OF THE INVENTION

The invention pertains to a polynucleotide for making plants more resistant to heat stress as well as to plants containing such a polynucleotide. The polynucleotide comprises a first sequence portion that is transcriptionally driving a second sequence portion, which is encoding a miRNA of the plant miRNA156 family.

### REFERENCE TO SEQUENCE LISTING

The present application is being filed along with a Sequence Listing in ASCII electronic format via EFS-web. The Sequence listing file, entitled 5535.21_{—} SEQ_{—} ASCII.txt, was created on Nov. 6, 2014 and is 10,862 bytes in size. The Sequence Listing is incorporated herein by reference in its entirety.

### BACKGROUND

To cope with changes in their physical environment, plants have evolved physiological responses allowing them to tolerate stressful conditions such as extreme temperatures, drought and high salinity (Jenks M A & Hasegawa P M (2005) Plant Abiotic Stress (Blackwell, Oxford)). These abiotic stresses are a major limiting factor for the geographical distribution and productivity of plants. Hence, stress tolerance is a central goal in crop breeding.

In plants, double-stranded RNA (dsRNA) is an effective trigger of RNA silencing, and several classes of endogenous small RNA (sRNA), processed from dsRNA substrates by endonucleases, are essential in controlling gene expression. One such sRNA class is the microRNAs (miRNAs).

MiRNAs are ca. 21 nucleotide long single-stranded RNA molecules that guide effector proteins, most commonly the ARGONAUTE1 (AGO1) protein, to complementary mRNA causing either mRNA cleavage or translational inhibition (Axtell M J (2013) Classification and Comparison of Small RNAs from Plants. Ann Rev Plant Biol 64:137-159; Voinnet O (2009) Origin, biogenesis, and activity of plant microRNAs. Cell 136(4):669-687). MiRNAs are generated from primary transcripts that are transcribed by RNA polymerase II and fold into a hairpin structure that is cleaved to release the active miRNA (Rogers K & Chen X (2013) Biogenesis, Turnover, and Mode of Action of Plant MicroRNAs. Plant Cell 25:2383-2399). MiRNAs play important roles during plant growth, developmental transitions and determination of cell identity (Voinnet O (2009) Origin, biogenesis, and activity of plant microRNAs. Cell 136(4):669-687; Rogers K & Chen X (2013) Biogenesis, Turnover, and Mode of Action of Plant MicroRNAs. Plant Cell 25: 2383-2399; Jones-Rhoades M W, Bartel D P, & Bartel B (2006) MicroRNAS and their regulatory roles in plants. Ann Rev Plant Biol 57:19-53). MiRNAs are also involved in the modulation of plant responses to biotic and abiotic stresses (Khraiwesh B, Zhu J K, & Zhu J (2012) Role of miRNAs and siRNAs in biotic and abiotic stress responses of plants. Biochim Biophys Acta 1819(2):137-148; Sunkar R, Li Y F, & Jagadeeswaran G (2012) Functions of microRNAs in plant stress responses. Trends Plant Sci 17(4):196-203; Kruszka K, et al. (2012) Role of microRNAs and other sRNAs of plants in their changing environments. J Plant Physiol 169(16):1664-1672; Fujii H, Chiou T J, Lin S I, Aung K, & Zhu J K (2005) A miRNA involved in phosphate-starvation response in Arabidopsis. Curr Biol 15(22):2038-2043; Navarro L, Jay F, Nomura K, He S Y, & Voinnet O (2008) Suppression of the microRNA pathway by bacterial effector proteins. Science 321(5891):964-967; Sunkar R & Zhu J K (2004) Novel and stress-regulated microRNAs and other small RNAs from Arabidopsis. Plant Cell 16(8):2001-2019).

One object of the present invention was to find a means of making plants more resistant to heat stress.

### BRIEF DESCRIPTION OF THE DRAWINGS

Exemplary embodiments of the present invention will be described below in more detail, with reference to the accompanying drawings, of which:

FIG. 1 shows a schematic overview of a method for providing a heat-stress resistant plant, according to embodiments of the invention.

FIG. 2 shows transcript levels of HS-responsive pri-miR156 and corresponding mature miRNA levels during maintenance of acquired thermotolerance according to embodiments of the invention.

FIG. 3 shows MiR156 regulating the maintenance of acquired thermotolerance and development according to embodiments of the present invention.

FIG. 4 shows MiR156 regulating the maintenance of acquired thermotolerance after HS according to embodiments of the present invention.

FIG. 5 shows further characterization of 35S::MIM156 and 35S::miR156h according to embodiments of the present invention.

FIG. 6 shows further characterization of heat-inducible miR156-manipulating lines according to embodiments of the present invention.

### DETAILED DESCRIPTION OF EXEMPLARY EMBODIMENTS OF THE INVENTION

Exemplary embodiments of the invention will now be discussed in further detail. This invention may, however, be embodied in many different forms and should not be construed as limited to the embodiments set forth herein.

The inventors discovered a function for miRNAs of the miR156 family in the heat stress (HS) response of plants. Members of the miR156 family are highly induced after HS and are functionally important for the HS memory. Members of the miR156 family promote sustained expression of a set of HS-responsive genes, and are critical only after HS, demonstrating that the effects of modulating miR156 levels on HS memory do not reflect pre-existing developmental alterations.

Accordingly, in a first aspect, the invention refers to a polynucleotide that can be used to generate heat-resistant plants (when compared to wild-type plants).

Such a polynucleotide comprises a first sequence portion that is able to transcriptionally drive a second sequence portion, wherein the first sequence portion is operably linked to a second sequence portion that encodes for a miRNA of the miRNA156 family. The first sequence portion is in one embodiment of the invention a heat-stress inducible promotor that allows for the transcription of a miRNA of the miRNA156 family, such as miRNA156, only after an initial heat stress. This reduces any possible negative effect that the expression of miRNA15 might have on the growth of the plant.

Since the first sequence portion is heat-stress inducible, it allows for the transcriptional activation of the second sequence portion, i.e. the transcription of a miRNA of the miRNA156 family, only after the plant was subject to heat stress.

The term “heat stress”, as used herein, refers to elevated growth temperatures that occur temporarily which are above the growth optimum temperature and cause potential denaturing of proteins and inhibition of physiological processes. A molecular marker of heat stress is the induction of heat shock protein (HSP) gene expression, as known in the art. HSP induction can be measured with any method suitable and known in the art, such as polymerase chain reaction (PCR) or Western blotting. Generally, temperatures above 33° C. are considered as heat stress. Time periods of at least 10 minutes may lead to heat stress.

The term “heat stress inducible promotor”, as used herein, refers to the expression of RNA from such a promotor that is induced only under heat stress. At the growth optimum temperature, RNA expression from a heat stress inducible promotor is not or only marginally detectable. Upon heat stress, the promotor becomes active and leads to the transcription of the sequence portion to which it is operably linked.

In one embodiment of the invention, the first sequence portion is a promotor selected from the group consisting of: HSP21, HSA32, HSP17, HSP18, HSP22, and HSP70. As examples, the sequences of the HSP21 and the HSP17 promotor of Arabidopsis thaliana are provided as SEQ ID NO. 8 and 9, respectively. The sequences of the other HSP promoters, also from other plants, are known in the art and can be retrieved, for example, from a suitable database. The coding sequences of heat shock proteins are highly conserved among different plants (Kotak S, Larkindale J, Lee U, von Koskull-Doring P, Vierling E, Scharf K D (2007) Complexity of the heat stress response in plants. Curr Opin Plant Biol 10: 310-316; Siddique M, Gernhard S, von Koskull-Doring P, Vierling E, Scharf K D (2008) The plant sHSP superfamily: five new members in Arabidopsis thaliana with unexpected properties. Cell stress & chaperones 13: 183-197; Scharf K D, Berberich T, Ebersberger I, Nover L (2012) The plant heat stress transcription factor (Hsf) family: structure, function and evolution. Biochimica et biophysica acta 1819: 104-119; Arazi T, Talmor-Neiman M, Stay R, Riese M, Huijser P, Baulcombe D C (2005) Cloning and characterization of micro-RNAs from moss. Plant J 43: 837-848; Axtell M J, Snyder J A, Bartel D P (2007) Common functions for diverse small RNAs of land plants. Plant Cell 19: 1750-1769; Cho S H, Coruh C, Axtell M J (2012) miR156 and miR390 regulate tasiRNA accumulation and developmental timing in Physcomitrella patens. Plant Cell 24: 4837-4849; Xin M, Wang Y, Yao Y, Xie C, Peng H, Ni Z, Sun Q (2010) Diverse set of microRNAs are responsive to powdery mildew infection and heat stress in wheat (Triticum aestivum L.). BMC plant biology 10: 123; Yu X, Wang H, Lu Y, de Ruiter M, Cariaso M, Prins M, van Tunen A, He Y (2012) Identification of conserved and novel microRNAs that are responsive to heat stress in Brassica rapa. J Exp Bot 63: 1025-1038).

The promotor may comprise regulatory sequences, such as enhancers and cis-elements.

The present invention can be practiced with all kind of plants. The sequences of miRNAs of the miRNA156 family are well-conserved among plant varieties and species. Therefore, a person of skill in the art can construct a polynucleotide with homologous sequences for the use in a particular plant variety or species based on the information provided herein together with his or her general knowledge in the art.

The term miRNA156 family, as used herein, refers to a polynucleotide in the form of the second sequence portion that is selected from the group consisting of: miRNA156a-f, miRNA156g, miRNA156h, and miR157.

Such polynucleotides are present in all plants, since miRNA156 family is well-conserved among different plant varieties and species.

The miRNA156 family also refers to functional fragments of the miRNAs listed above. A functional fragment is a polynucleotide that is shorter than miRNA156a-f, miRNA156g, miRNA156h, and miR157, respectively, but exerts the same physiological function in making a plant heat resistant.

In plants, a single miRNA small RNA (sRNA) silencing signal is processed from a long precursor transcript of nonprotein-coding RNA, termed the primary miRNA (pri-miRNA). A region of the pri-miRNA is partially self-complementary allowing the transcript to fold back onto itself to form a stem-loop structure of (possibly imperfectly) double-stranded RNA (dsRNA).

Accordingly, in one embodiment, the second sequence portion comprises or consists of a natural immature miRNA sequence that allows for the formation a secondary structure, in particular a stem-loop structure, of the transcript.

Artificial miRNA (amiRNA) technology uses endogenous pri-miRNAs, in which the miRNA and miRNA* (a passenger strand of the miRNA duplex) sequences have been replaced with corresponding amiRNA/amiRNA* sequences that direct highly efficient RNA silencing of the targeted gene (Ossowski, S., Schwab, R., and Weigel, D., Gene silencing in plants using artificial microRNAs and other small RNAs. Plant J. 2008; 53(4): 674-690; Eamens, A. L., McHale, M., and Waterhouse, P. M. The Use of Artificial MicroRNA Technology to Control Gene Expression in Arabidopsis thaliana. Methods Mol Biol. 2014; 1062:211-224).

Accordingly, in certain embodiments of the invention, the second sequence portion may also be, at least in part, artificially engineered.

Put differently, in one embodiment of the invention, the second sequence portion comprises a sequence that encodes a mature miRNA sequence that does not form a secondary structure by itself. In this case, the second sequence portion comprises sequences that allow for the formation of a stem-loop structure. Such a stem-loop structure is advantageous for the correct processing and localization of the miRNA within the plant cell. The sequences that allow for the formation of a stem-loop structure can be natural or artificial.

Examples of polynucleotides of the invention are combinations of SEQ ID NO. 8 operably linked to a sequence of SEQ ID NO. 1, SEQ ID NO. 2, SEQ ID NO. 3, SEQ ID NO. 4, SEQ ID NO. 5, SEQ ID NO. 6, or SEQ ID NO. 7. Further examples of polynucleotides of the invention are combinations of SEQ ID NO. 9 operably linked to a sequence of SEQ ID NO. 1, SEQ ID NO. 2, SEQ ID NO. 3, SEQ ID NO. 4, SEQ ID NO. 5, SEQ ID NO. 6, or SEQ ID NO. 7.

The polynucleotide is selected preferably from the group consisting of desoxyribo nucleic acid (DNA), and chemically modified, i.e. artificial DNAs such as Peptide Nucleic Acid (PNA) or morpholinos.

The backbone of PNAs is composed of repeating N-(2-aminoethyl)-glycine units linked by peptide bonds. The various purine and pyrimidine bases are linked to the backbone by a methylene bridge (—CH₂—) and a carbonyl group (—(C═O)—) (Nielsen P E, Egholm M, Berg R H, Buchardt O (December 1991). Sequence-selective recognition of DNA by strand displacement with a thymine-substituted polyamide. Science 254 (5037): 1497-500).

Morpholinos have standard nucleic acid bases that are bound to morpholine rings instead of deoxyribose rings and linked through phosphorodiamidate groups instead of phosphates (Summerton, J; Weller D (1997). “Morpholino Antisense Oligomers: Design, Preparation and Properties”. Antisense & Nucleic Acid Drug Development 7 (3): 187-95).

In another aspect, the invention refers to a vector (i.e. a small piece of DNA that can be stably maintained in an, and into which a polynucleotide of the invention can be inserted for cloning and expression purposes) comprising a polynucleotide as described above and herein. Such a vector may be a plasmid, a cosmid or an artificial chromosome. The vector can be used to provide a polynucleotide of the invention in a living plant cell, in a plant tissue or a whole plant. The vector may generate a miRNA of the miRNA156 family upon heat-stress in cells of the plant.

The vector may remain episomal in a plant cell, or integrates into the genome of the plant cell.

Expression constructs of the invention may optionally contain a transcription termination sequence and/or enhancer elements. Transcription termination sequences can be positioned downstream of a coding sequence to provide for efficient termination. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element.

The vector of the invention can be replicated and isolated using any method known in the art.

In another aspect, the invention refers to a heat-resistant plant cell (i.e. a single cell, isolated or as part of a group of cells, comprising plant tissues or whole plants), which comprises a polynucleotide as described above and herein.

Such a heat resistant plant cell comprises at least one polynucleotide as described above and herein, comprising a heat-inducible promotor operably linked to a sequence of the miRNA156 family.

In another aspect, the invention refers to a method for generating a heat-resistant plant. Such a method comprises the step of introducing at least one polynucleotide as described above and herein, with a HS-inducible promotor upstream of a sequence of the miRNA156 family and operably linked thereto, into a plant cell.

The introduction into the plant cell can be performed using any suitable method known in the art. Techniques for transforming plant cells with a gene include, for example, Agrobacterium infection, biolistic methods, electroporation, calcium chloride treatment, PEG-mediated transformation, etc. Transformed cells can be selected, redifferentiated, and grown into plants that contain and express a polynucleotide of the invention using standard methods known in the art. The seeds and other plant tissue and progeny of any transformed or transgenic plant cells or plants of the invention are also included within the scope of the present invention.

The subject invention also concerns methods for producing a plant that exhibits increased heat stability relative to a wild type plant, wherein a polynucleotide of the invention, encoding a miRNA of the miRNA156 family under the control of a heat-inducible promotor is introduced into a plant cell and the polypeptide(s) encoded by the polynucleotide(s) is expressed.

In yet another aspect, the invention refers to a method of increasing resistance of a plant to heat stress conditions comprising the step of introducing a polynucleotide as described above and herein into a plant cell.

In an alternative embodiment, it is also possible to generate a polynucleotide as described above and herein in a plant cell. For example, a sequence of the miRNA156 family in the form of a stem-loop-precursor can be introduced at the 3′ side of an endogenous promotor, such that promotor and miRNA are operably linked and the miRNA can be expressed from the endogenous heat-inducible promotor. Alternatively, it is possible to introduce a heat-inducible promotor at the 5′ side of an endogenous miRNA member of the miRNA156 family such that the endogenous miRNA is under the transcriptional control of the introduced promotor.

The replacement of endogenous sequences can be achieved using methods known in the art, which are based on homologous recombination.

In another aspect, the invention pertains to a method for generating a heat-resistant plant, as described herein, comprising the step of generating a polynucleotide as described herein in a plant cell or of introducing a polynucleotide as described herein into a plant cell.

In a further aspect, the invention pertains to a kit for generating a heat-resistant plant. Such a kit may comprise a vector comprising a polynucleotide of the invention, optionally together with other components, e.g., oligonucleotide probes and primers, nucleotides, buffer solutions, etc.

The oligonucleotide probes and primers can be polymerase chain reaction (PCR) primers that can hybridize to the polynucleotide of the invention. Oligonucleotide probes of the invention can be used in methods for detecting and quantitating nucleic acid sequences encoding a miRNA of the miRNA156 family. Oligonucleotide primers of the invention can be used in PCR methods and other methods involving nucleic acid amplification. Probes and primers of the invention can optionally comprise a detectable label or reporter molecule, such as fluorescent molecules, enzymes, radioactive moiety (e.g., 3H, 35S, 125I, etc.), and the like. Probes and primers of the invention can be of any suitable length for the method or assay in which they are being employed. Typically, probes and primers of the invention will be 10 to 500 or more nucleotides in length.

The invention also refers to the use of such a kit for generating a heat-resistant plant.

### Sequences

ath-miR156a
SEQ ID NO. 1: miRNA156a-f
TGACAGAAGAGAGTGAGCAC
ath-miR156g
SEQ ID NO. 2: miRNA156g
CGACAGAAGAGAGTGAGCAC
ath-miR156h
SEQ ID NO. 3: miRNA156h
TGACAGAAGAAAGAGAGCAC
ath-miR157a
SEQ ID NO. 4: miRNA156a
TTGACAGAAGATAGAGAGCAC
ath-miR157b
SEQ ID NO. 5: miRNA156b
TTGACAGAAGATAGAGAGCAC
ath-miR157c
SEQ ID NO. 6: miRNA156c
TTGACAGAAGATAGAGAGCAC
ath-miR157d
SEQ ID NO. 7: miRNA156d
TGACAGAAGATAGAGAGCAC
AthHSP21 promotor sequence
SEQ ID NO. 8: HSP21
TCAAGAAATTAGGGTTCCCAGATGAAAAAGAGGATATGTTAGCAGAAG
ACGGAAACAAATTTGGTTGTTCTTGTGGACGACGACAGAGCTTTTGCC
TTGGTGGGGTTTTCTCGAATACTTGGAGATCGAGGAAAGGAGAAGCTG
AAATTACCGAAATAGCCTTAGCTGCACCGTCGTAAGTCGTGTAACTAG
ATTGTGTCAAGAAGGGCCTTTAATAGGCCTTCAATTCAGAATGTTCTA
AAACCGAATTAAAGTACTGTTCGGCGTCCACTAGATTTTGTAGGTACT
ATTGCATCATCATCATCTTATATCAGTTCAATGTGTGTCTTGTTTTTT
CTTTTTTAGTCTATTACATCATCTGATCTTATTCTAGCAGCCTTTTGA
AAATATAGAGAAAAATCATTCAATGTTTCTAAAACCAAATCTATACAC
AAAACATAAAGTCTTTTGGATCATTGAACATTTGATCGGATTGCGGAT
TAATAGATTAGAGCTTAAATTTTGTATAATTTTATACCAATCAAAATT
AATATAGAGTCATGTCATGCTATTTGTATAATTTATGTACAACAATTA
TTATATAAAACGACTTGAACAATTCTGTGATAAAGAAAAAAAAAAAAA
AAACGACTTGAACAATTCAACATGCTATAGATAGTTTATACGTAAGCT
AATAAGCCTGTTTGATACTGTATATGACATACGTGTAGCTGATATGAT
CAAACTAGACAAAATTTTCTAATTTCTCCCGTAGATATATATTTTTTA
GTTTACTTGGGCATTCCATTTCTCATATGTTTCTGCAAGTTAGCACAC
ATAATGGTGACTTGGTGAGTTGCTTTTTTCTCTCTCTTTTTCAATATG
ATATGCTTTCTTCGATTTTTTTATTTTTTHAAACAATATATCATCTAA
TCATGGTTTTTTCGTAATCGTGGTTTTTCTAATTGTAGAAATCTAGTC
TTTTTTTTTCGTTTATTTTGTTTTTATTAGTTCATCACAAATAAACAC
TGATTGGCAAGAAGGAAAAAAAGAAGATGGTTCGGGAGAAGATGAGAA
AACACGAGAAAACGCACAAAGGTTAACAAATAGTAAAATAGATAGATA
GAAAACAAAACGAGTCGAGAAACTTCGTATCAAATCTTCAACCTTCAA
GACCTCTCCTTTTGTCACTTAGAGCCTTCTCATCCTCACCTACATATA
TACACTCACTTCTACAATTGTAGTAGATACTCAACAAACTCAGAACTA
GAATTTTCTAATCTTCACTCAGCGATATTTTTGGCTCATACTCGAAAC
AA
Hvu HSP17 promotor sequence
SEQ ID NO.9: HSP17
GTAGAGAATTTTAAGATGTCCATGACCACCCGACCACCCGCTAGCTGC
GTCATTGTGTAGTACTCTTTCTGTTTTTAAAATAAGTGTCTGAACTTT
ATACTAACTTTAATATAAAATTGTACTAAACTTGAGACATTTATTTAG
ACGGAGGGAGTAGTATTTATTTACGTTTAACTATTGTACTTGCTCGCT
GTAAACTTTAATGACAGATAACCTCTACTGTTAATCATGCTATTCTGG
AAACGGCCCCACAACCCATTCTTGAATCTAGACTAGTCGCCCCCTTGC
TGGAATTATACAATTATTTATAGAAAAATCTCACAAAAAAATTATTAA
CAAAAAAAAAAGGGAGAAGGCGCCCTAACGGCAGGTTCGTGAGTCCCC
TGGAAGACGATAGACCCTTCGAGGGTTGGGAACAGGAAAGAACGAATG
GCTCACAGCCTACCTGGAAAGCGAAAGAGAAACAAACACTCGTGGATT
GGACAGTCCTCGAGATTCTTCAAGATCAGTACCTACGATGCTATAAGT
AGTGGCTCACCTTCCATCGCCCTCCCAACTCAAGCCAGCCAGCGTCAA
ACCAAAGCGAACAACATCCTACCTACAATCCGATACAGATTAATTTCA
ACGCGAGCTACAAACCAAAGCAAGCAACACCGACA
hvu-MIR156
SEQ ID NO. 10: Stem loop sequence Barley
(Hordeum vulgare)
UGACAGAAGAGAGUGAGCACACAGCGGCAGGCUACGUCUGAUAAGAGC
AUGGUUAAUAGUAUAGCCAACUGCUGGCUAUACUAUGAGCACGUGCUU
AGACGCAGCUGCUGUUGCGUGCUCACCCUCUCUCUGUCU
hvu-miR156
SEQ ID NO. 11: mature miRNA Barley
(Hordeum vulgare)
UGACAGAAGAGAGUGAGCACA
bna-miR156a
SEQ ID NO. 12: mature miRNA Brassica napus
UGACAGAAGAGAGUGAGCACA

### Examples

### Materials and Methods

Plant Material and Growth Conditions Plants were grown in GM medium (1% (w/v) glucose) at 23° C./21° C. 16 h light/8h dark cycles unless otherwise indicated. ago1-25, ago1-27, and dcl1-9 were obtained from N. Baumberger and C. Dean, respectively. SPL9::SPL9-GUS, SPL9::rSPL9-GUS and suo-2 (Yang L, Wu G, & Poethig R S (2012) Mutations in the GW-repeat protein SUO reveal a developmental function for microRNA-mediated translational repression in Arabidopsis. Proc Natl Acad Sci USA 109(1):315-320) were obtained from S. Poethig. 35S::MIM156 and 35S::MIMIPS1 Agrobacterium stocks, hsp101 (N566394), hsfa2 (N508978) were obtained from the Nottingham Arabidopsis Stock Centre. Heat stress treatments were performed in a waterbath or incubator. For the priming HS (ACC), 3-4d old seedlings were subjected to a heat regime of 1 h 37° C., 1.5 h 23° C., 45 min 44° C., except where a short ACC was applied omitting the 44° C. step. A tester HS was applied 2-3 d later for 45-110 min. For acquisition of thermotolerance the regime was 1 h 37° C., 1.5 h 23° C., 0-180 min 44° C.; basal thermotolerance was assayed by incubation for 0-60 min at 44° C. After heat treatment, plants were returned to normal growth conditions until analysis. For analysis of gene expression during maintenance of acquired thermotolerance, 3-4 d old seedlings were subjected to ACC or mock treatment and harvested at the indicated time points.

### Construction of Transgenic Lines

For SPL2::rSPL2, a 3.9 kb promoter fragment was amplified with primers (all primer sequences are given in Table 1 and in the sequence listing) introducing NcoI and SacI restriction sites. The coding sequence and 3′ region (2.5 kb) was amplified mutating the miRNA binding site while preserving the amino acid sequence with primers introducing SacI and XmaI sites (Wang J W, Schwab R, Czech B, Mica E, & Weigel D (2008) Dual effects of miR156-targeted SPL genes and CYP78A5/KLUH on plastochron length and organ size in Arabidopsis thaliana. Plant Cell 20(5):1231-1243). After sequencing, the SPL2::rSPL2 construct was assembled in pUC-ML939 and transferred via AscI into pBarMAP-ML516. For SPL11::rSPL11, a 2.6 kb promoter fragment was amplified with primers introducing NcoI and SacI restriction sites. The coding sequence and 3′ region (2.5 kb) was amplified mutating the miRNA binding site while preserving the amino acid sequence with primers introducing SacI and XmaI sites. After sequencing, the SPL11::rSPL11 construct was assembled in pUC-ML939 and transferred via AscI into pBarMAP-ML516. Transgenic plants were generated using the floral dip method (Clough S J & Bent A F (1998) Floral dip: a simplified method for Agrobacterium-mediated transformation of Arabidopsis thaliana. Plant J 16(6):735-743). For 35S::MIR156h, a 0.5 kb fragment encompassing the pri-miR156h sequence was amplified introducing XhoI and BamHI restriction sites and subcloned in ML595. MIM156h was generated using specific primer sequences analogous to MIM156 (Franco-Zorrilla J M, et al. (2007) Target mimicry provides a new mechanism for regulation of microRNA activity. Nature Genet 39(8):1033-1037). HSP21::MIR156h and HSP21::MIM156h were generated by replacing the HSP21 coding sequence in a 2.8 kb genomic fragment with the MIR156h and MIM156h fragments described above. All experiments were performed with multiple homozygous lines carrying a single insertion (determined by segregation).

RNA Preparation, Microarray Analysis and qRT-PCR

RNA was extracted from seedlings using a Trizol-based protocol (Pant B D, et al. (2009) Identification of nutrient-responsive Arabidopsis and rapeseed microRNAs by comprehensive real-time polymerase chain reaction profiling and small RNA sequencing. Plant Physiol 150(3):1541-1555) or a hot-phenol RNA extraction protocol (Bäurle I, Smith L, Baulcombe D C, & Dean C (2007) Widespread role for the flowering-time regulators FCA and FPA in RNA-mediated chromatin silencing. Science 318(5847): 109-112). For microarray analysis, RNA from three biological replicates per genotype and treatment (4h after ACC, 52 h after ACC, 4 h NTC) was purified over an RNeasy Plant RNA extraction column (Qiagen), and processed for hybridization of Affymetrix ATH1 Genechips (NASC microarray facility). For quantitative RT-PCR, total RNA was treated with TURBO DNA-free (Ambion) and 2 μg reverse transcribed with SuperScript III (Invitrogen) according to the manufacturers' instructions. 0.1 μl cDNA was used per 10 μl QPCR reaction with GoTaq qPCR Master Mix (Promega) and an Applied Biosystems AB7300 or Roche LightCycler 480 instrument. Expression was normalized to TUBULIN6 using the comparative CT method. Primer sequences are listed in Table 1 and in the sequence listing.

Pri-miRNA Profiling, miRNA Target Prediction and Mature miRNA Detection

The pri-miRNA profiling was performed as described (Pant B D, et al. (2009) Identification of nutrient-responsive Arabidopsis and rapeseed microRNAs by comprehensive real-time polymerase chain reaction profiling and small RNA sequencing. Plant Physiol 150(3):1541-1555). The miRNA target prediction was performed using online search tools with the default settings (Ossowski S, Schwab R, & Weigel D (2008) Gene silencing in plants using artificial microRNAs and other small RNAs. Plant J 53(4):674-690; Dai X & Zhao P X (2011) psRNATarget: a plant small RNA target analysis server. Nuc Acids Res 39 (Web Server issue):W155-159; Moxon S, et al. (2008) A toolkit for analysing large-scale plant small RNA datasets. Bioinformatics 24(19):2252-2253; Alves L, Jr., Niemeier S, Hauenschild A, Rehmsmeier M, & Merkle T (2009) Comprehensive prediction of novel microRNA targets in Arabidopsis thaliana. Nuc Acids Res 37(12): 4010-4021) and published degradome data (39). For detection of mat-miRNA, a modified stem-loop qRT-PCR protocol was used with SYBR Green as a dye (60, 61).

MUG Assay To quantify GUS protein level, 4-Methylumbelliferyl b-D-glucuronidase (MUG) activity was determined with a fluorescence spectrophotometer according to standard procedures (Jefferson R A, Kavanagh T A, & Bevan M W (1987) GUS fusions: beta-glucuronidase as a sensitive and versatile gene fusion marker in higher plants. EMBO J 6(13):3901-3907).

TABLE 1
Sequences of oligonucleotides

| SEQ | | | | |
|---|---|---|---|---|
| ID | | | | Appli- |
| NO | Gene | Name | Sequence | cation |
| 13 | | 435/miR156e_F | GTCACATGCGTA | qRT-PCR |
| | | | GAGTGTGAAAGG | pri-miRNA |
| 14 | | 436/miR156e_R | CCATGTGTGCTC | qRT-PCR |
| | | | ACTCTCTTCTGT | pri-miRNA |
| 15 | | 437/miR156h_F | TCCTCCCGAGAA | qRT-PCR |
| | | | GAAGATGAAAA | pri-miRNA |
| 16 | | 438/miR156h_R | GCTAATCCCAGG | qRT-PCR |
| | | | TTGTGCTCTCT | pri-miRNA |
| 17 | | atmiR156gStLRT | GTCGTATCCAGT | qRT-PCR |
| | | | GCAGGGTCCGAG | |
| | | | GTATTCGCACTG | |
| | | | GATACGACGTGC | |
| | | | T | |
| 18 | | atmiR156gTqmn_F | TCGACGCGACAG | qRT-PCR |
| | | | AAGAGAGTGA | |
| 19 | | miR156hStLpRT | GTCGTATCCAGT | qRT-PCR |
| | | | GCAGGGTCCGAG | |
| | | | GTATTCGCACTG | |
| | | | GATACGACGTGC | |
| | | | TC | |
| 20 | | miR156hTqMn_F | CGTCGACGTGAC | qRT-PCR |
| | | | AGAAGAAAGA | |
| 21 | MIM | 555/IPS1_F | CTCGAGAAGAAA | Franco- |
| | | | AATGGCCATCCC | Zorilla |
| | | | CTAGC | et al. |
| | | | | 2007 |
| 22 | MIM | 556/IPS1_R | GGATCCGAGGAA | Franco- |
| | | | TTCACTATAAAG | Zorilla |
| | | | AGAATCG | et al. |
| | | | | 2007 |
| 23 | MIM | 557/ | CTTGACAGAAGA | MIM156h |
| | | MIM156h_I | TAGGAGAGAGCA | |
| | | | TTTTCTAGAGGG | |
| | | | AGATAA | |
| 24 | MIM | 558/ | AAATGCTCTCTC | MIM156h |
| | | MIM156h_II | CTATCTTCTGTC | |
| | | | AAGCTTCGGTTC | |
| | | | CCCTCG | |
| 25 | miR156h | 465/156hoxF | ACTCGAGATAGC | MIR156h |
| | | | GAAGAGGTAAGA | |
| | | | AAGTG | |
| 26 | miR156h | 466/156hoxR | AGGATCCAAAAC | MIR156h |
| | | | CCAATTTCAAGA | |
| | | | TTGATC | |
| 27 | pri- | 1192/pri- | CTGACAGAAGAG | qRT-PCR |
| | miR156c | miR156c_F | AGTGAGCACACA | |
| | | | AAG | |
| 28 | pri- | 1193/pri- | CAGATAGAGCAG | qRT-PCR |
| | miR156c | miR156c_R | TGAGCACGCAAG | |
| 29 | pri- | 1194/pri- | CAGAAGAGAGTG | qRT-PCR |
| | miR156d | miR156d_F | AGCACACAAAGG | |
| | | | G | |
| 30 | pri- | 1195/pir- | GTGAGCACGCAA | qRT-PCR |
| | miR156d | miR156d_R | AAGCAACCATAT | |
| | | | AC | |

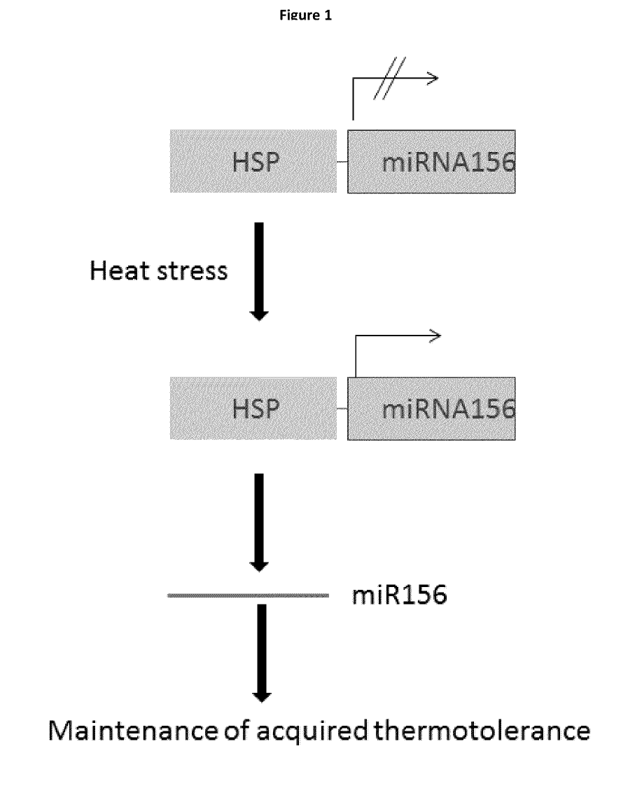

FIG. 1 depicts the principle of the invention. A member of the miRNA156 family of miRNAs is positioned such that it is under the control of a heat-stress inducible promotor. After introduction into a plant cell, the member of the miRNA156 family of miRNAs is expressed upon heat stress (miRNA156 is shown here as an example), rendering the plant heat-resistant.

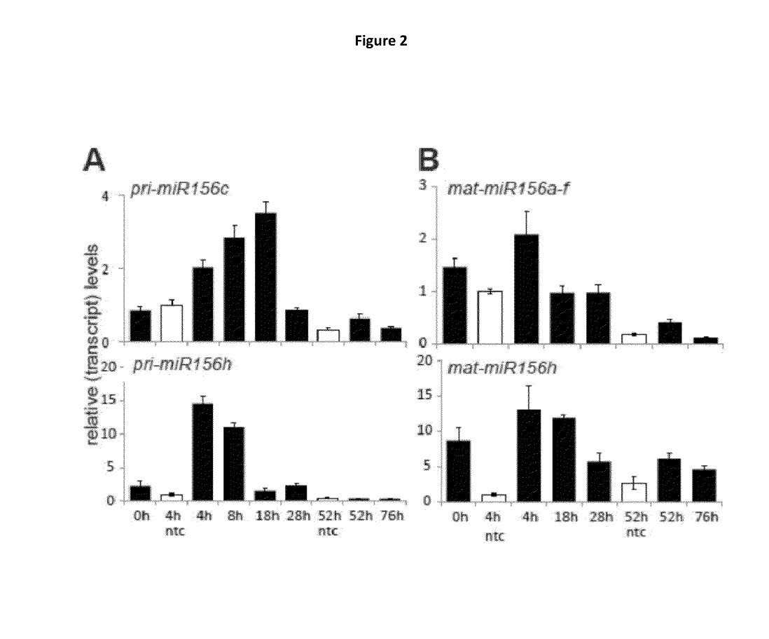

In FIG. 2, (A) corresponds to quantitative RT-PCR for pri-miR156c and pri-miR156h; and (B) corresponds to quantitative RT-PCR for mature miR156a-f and miR156h.

Expression values were normalized to TUB6 and the respective 4 h ntc. Error bars are SEM of three replicates. One representative of at least three independent experiments is shown.

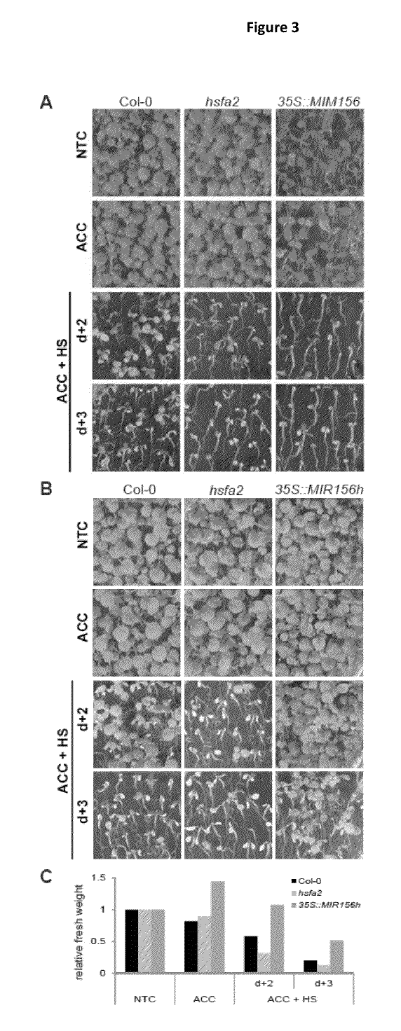

In FIG. 3, (A) corresponds to knockdown of miR156a-f in 35S::MIM156 impairs HS memory, assayed by applying a tester HS (HS) two (+2) and three days (+3) after a priming HS (ACC). Pictures were taken 14 d after ACC. All plants of one treatment were grown on the same plate. (B) corresponds to overexpression of miR156h enhances the HS memory, assayed by applying a tester HS (HS) two (+2) and three days (+3) after a short ACC. Pictures were taken 14 d after ACC. All plants of one treatment were grown on the same plate. (C) corresponds to quantification of (B); average seedling fresh weight was determined from 27±1 seedlings per genotype and treatment.

The experiments were repeated at least 5 times with at least two independent transgenic lines with similar results.

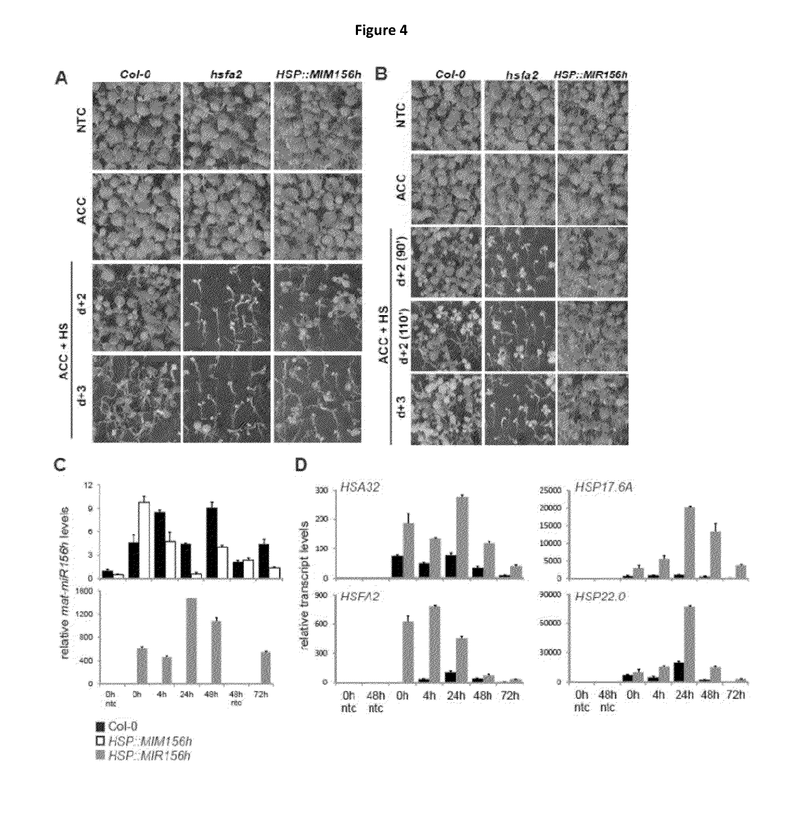

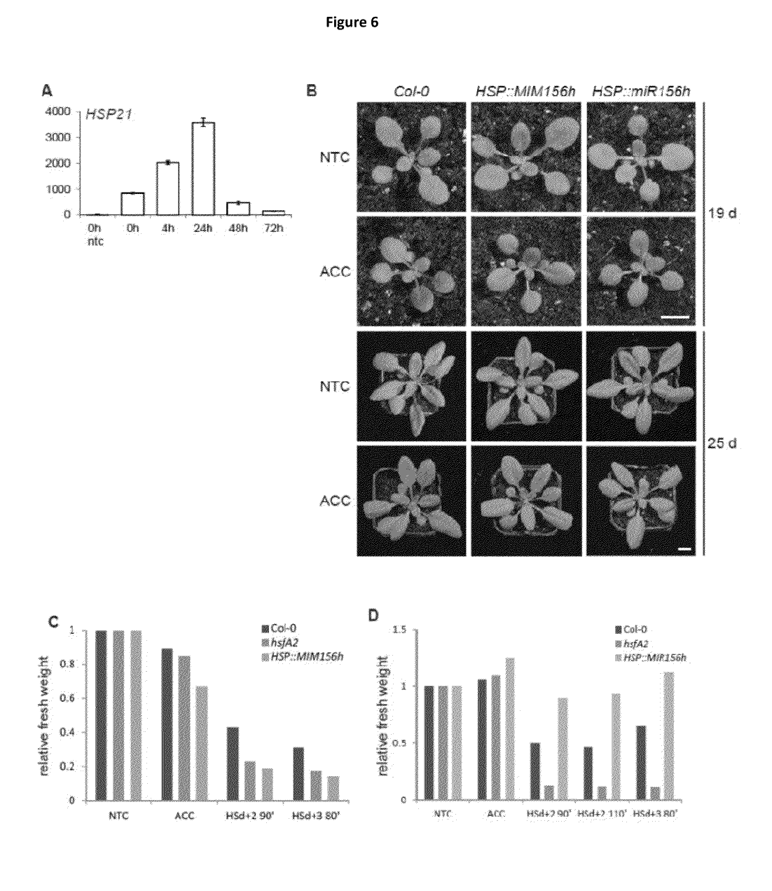

In FIG. 4, (A) corresponds to heat-inducible knock-down of miR156h (HSP21::MIM156h) impairs the HS memory, assayed by applying a tester HS (HS) two (+2) and three days (+3) after a priming HS (ACC). Pictures were taken 14 d after ACC. All plants of one treatment were grown on the same plate. See FIG. 6C for quantification. (B) corresponds to heat-inducible overexpression of MIR156h (HSP21::MIR156h) enhances the HS memory, assayed by applying a tester HS (HS) two (+2, 90, 110 min) and three days (+3) after a short ACC. Pictures were taken 14 d after ACC. All plants of one treatment were grown on the same plate. See FIG. 6D for quantification. (C) corresponds to mat-miRNA levels of miR156h in HSP::MIR156h and HSP::MIM156h after ACC determined by qRT-PCR. Error bars are SEM of three replicates. Three independent experiments were performed with one representative shown. (D) corresponds to expression of selected HS-memory related genes determined by qRT-PCR is increased after a priming HS in HSP::MIR156h. Expression values were normalized to TUB6 and 48 h ntc. Error bars are SEM of three replicates. Three independent experiments were performed with one representative shown.

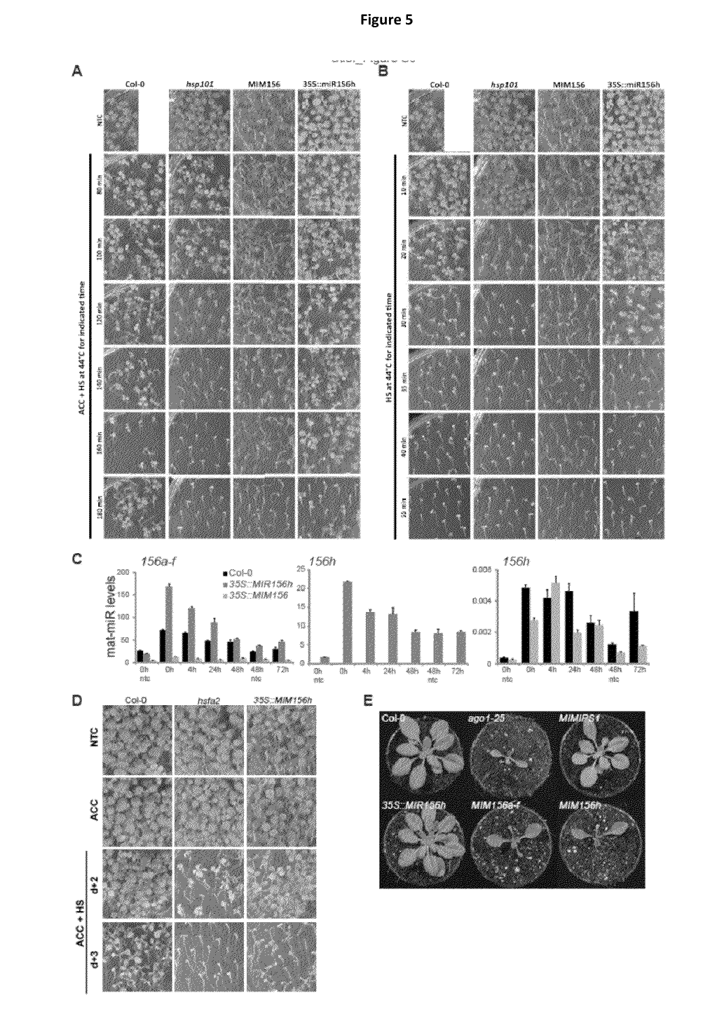

In FIG. 5, (A) corresponds to acquisition of thermotolerance in 35S::MIM156a-f and 35S::MIR156h was assayed by extending the second HS of the priming HS treatment for the indicated times. Pictures were taken 8 d after the HS. (B) corresponds to basal thermotolerance was assayed by incubating 4 day old seedlings for the indicated times at 44° C. Pictures were taken 8 d after the HS. (C) corresponds to mat-miRNA levels of miR156a-f and miR156h in Col (black), 35S::MIR156h (blue) and 35S::MIM156 (green) determined by qRT-PCR. Error bars are SEM of three replicates. The experiment was repeated at least twice independently and one representative is shown. (D) corresponds to 35S::MIM156h plants were assayed for the maintenance of acquired thermotolerance by applying a tester HS (HS) two (+2, 90 min) and three days (+3, 90 min) after a priming HS (ACC). Pictures were taken 14d after ACC. (E) corresponds to effects of 35S::MIM156a-f, 35S::MIM156h and 35S::miR156h on rosette phenotypes of 22 d old plants grown in long days. Size bar is 1 cm. MIMIPS1 [Fornara F, de Montaigu A, & Coupland G (2010) SnapShot: Control of flowering in Arabidopsis. Cell 141(3):550, 550 e551-552] was included as a control.

All plants of one treatment were grown on the same plate. The result is representative for several experiments with homozygous lines derived from multiple independent transformants.

In FIG. 6, (A) corresponds to transcript levels of HSP21 after a priming HS as determined by qRT-PCR normalized to TUB6 and 0 h ntc. (B) corresponds to 19 and 25 day-old rosette plants of the indicated genotypes treated with a priming HS (ACC) on day 4 or mock-treated (NTC). Size bars are 1 cm. (C) corresponds to quantification of FIG. 4A; average seedling fresh weight was determined from 32±3 seedlings per genotype and treatment. (D) corresponds to quantification of FIG. 6B; average seedling fresh weight was determined from 24±3 seedlings per genotype and treatment.

Although illustrative embodiments of the present invention have been described herein with reference to the accompanying drawings, it is to be understood that the invention is not limited to those precise embodiments, and that various other changes and modifications may be made by one skilled in the art without departing from the scope or spirit of the invention.

1-15. (canceled)

16. A polynucleotide, comprising:
a first sequence portion for driving transcription of a second sequence portion; and
a second sequence portion encoding a miRNA of plant miRNA156 family;
wherein the first sequence portion is heat-stress inducible and operably linked to the second sequence portion.

17. The polynucleotide of claim 16, wherein the first sequence portion is a promotor selected from the group consisting of HSP21, HSA32, HSP17, HSP18, HSP22, and HSP70.

18. The polynucleotide of claim 16, wherein the miRNA of the plant miRNA156 family comprises a sequence selected from the group consisting of miRNA156a-f, miRNA156g, miRNA156h, and miR157a-d.

19. The polynucleotide of claim 16, wherein the polynucleotide is one of a DNA, an RNA, a chemically modified DNA, and a chemically modified RNA.

20. The polynucleotide of claim 16, wherein the polynucleotide is derived from a plant selected from a monocotyledonous plant and a dicotyledonous plant.

21. The polynucleotide of claim 20, wherein the monocotyledonous plant is selected from the group consisting of rice, wheat, barley, oats, rye, sorghum, maize, sugarcane, pineapple, onion, bananas, coconut, lilies, turfgrasses, and millet.

22. The polynucleotide of claim 20, wherein the dicotyledonous plant is selected from the group consisting of tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus, grape, cotton, sunflower, strawberry, lettuce, and hop.

23. A vector comprising the polynucleotide of claim 16, wherein the vector is one of a plasmid, an artificial chromosome, and a cosmid.

24. A heat-resistant plant, comprising a polynucleotide of claim 16.

25. The heat-resistant plant of claim 24, wherein the polynucleotide is located chromosomally or exrachomosomally.

26. A heat-resistant plant cell comprising a polynucleotide, the polynucleotide comprising:
a first sequence portion for driving transcription of a second sequence portion; and
a second sequence portion encoding a miRNA of plant miRNA156 family;
wherein the first sequence portion is heat-stress inducible and operably linked to the second sequence portion.

27. The heat-resistant plant cell of claim 26, wherein the polynucleotide is located chromosomally or exrachomosomally.

28. The heat-resistant plant cell of claim 26, wherein the polynucleotide is derived from a plant selected from a monocotyledonous plant and a dicotyledonous plant.

29. The heat-resistant plant cell of claim 28, wherein the monocotyledonous plant is selected from the group consisting of rice, wheat, barley, oats, rye, sorghum, maize, sugarcane, pineapple, onion, bananas, coconut, lilies, turfgrasses, and millet.

30. The heat-resistant plant cell of claim 28, wherein the dicotyledonous plant is selected from the group consisting of tomato, cucumber, squash, peas, alfalfa, melon, chickpea, chicory, clover, kale, lentil, soybean, beans, tobacco, potato, sweet potato, yams, cassava, radish, broccoli, spinach, cabbage, rape, apple trees, citrus, grape, cotton, sunflower, strawberry, lettuce, and hop.

31. A method for generating a heat-resistant plant, comprising at least one of generating a polynucleotide in a plant cell and introducing the polynucleotide into a plant cell, the polynucleotide comprising:
a first sequence portion for driving transcription of a second sequence portion; and
a second sequence portion encoding a miRNA of plant miRNA156 family;
wherein the first sequence portion is heat-stress inducible and operably linked to the second sequence portion.

32. The method of claim 31, wherein the first sequence portion is a promotor selected from the group consisting of HSP21, HSA32, HSP17, HSP18, HSP22, and HSP70.

33. The method of claim 31, wherein the miRNA of the plant miRNA156 family comprises a sequence selected from the group consisting of miRNA156a-f, miRNA156g, miRNA156h, and miR157a-d.

34. The method of claim 31, further comprising subjecting the plant to a heat stress.

35. The method claim 31, further comprising:
selecting the plant; and
integrating the polynucleotide into the genome of the plant.


SEQUENCE LISTING (30 sequences)

SEQ ID NO: 1 - DNA, 20 bp, Arabidopsis thaliana
  source 1..20: /organism=“Arabidopsis thaliana” /mol_type=“unassigned DNA”
tgacagaagagagtgagcac

SEQ ID NO: 2 - DNA, 20 bp, Arabidopsis thaliana
  source 1..20: /organism=“Arabidopsis thaliana” /mol_type=“unassigned DNA”
cgacagaagagagtgagcac

SEQ ID NO: 3 - DNA, 20 bp, Arabidopsis thaliana
  source 1..20: /organism=“Arabidopsis thaliana” /mol_type=“unassigned DNA”
tgacagaagaaagagagcac

SEQ ID NO: 4 - DNA, 21 bp, Arabidopsis thaliana
  source 1..21: /organism=“Arabidopsis thaliana” /mol_type=“unassigned DNA”
ttgacagaagatagagagcac

SEQ ID NO: 5 - DNA, 21 bp, Arabidopsis thaliana
  source 1..21: /organism=“Arabidopsis thaliana” /mol_type=“unassigned DNA”
ttgacagaagatagagagcac

SEQ ID NO: 6 - DNA, 21 bp, Arabidopsis thaliana
  source 1..21: /organism=“Arabidopsis thaliana” /mol_type=“unassigned DNA”
ttgacagaagatagagagcac

SEQ ID NO: 7 - DNA, 20 bp, Arabidopsis thaliana
  source 1..20: /organism=“Arabidopsis thaliana” /mol_type=“unassigned DNA”
tgacagaagatagagagcac

SEQ ID NO: 8 - DNA, 1299 bp, Arabidopsis thaliana
  source 1..1299: /organism=“Arabidopsis thaliana” /mol_type=“unassigned DNA”
tcaagaaattagggttcccagatgaaaaagaggatatgttagcagaagacggaaacaaatttggttgttcttgtggacgacgacagagcttttgccttggtgggggtttctcgaatacttggagatcgaggaaaggagaagctgaaattaccgaaatagccttagctgcaccgtcgtaagtcgtgtaactagattgtgtcaagaagggcctttaataggccttcaattcagaatgttctaaaaccgaattaaagtactgttcggcgtccactagattttgtaggtactattgcatcatcatcatcttatatcagttcaatgtgtgtcttgttttttcttttttagtctattacatcatctgatcttattctagcagccttttgaaaatatagagaaaaatcattcaatgtttctaaaaccaaatctatacacaaaacataaagtcttttggatcattgaacatttgatcggattgcggattaatagattagagcttaaattttgtataattttataccaatcaaaattaatatagagtcatgtcatgctatttgtataatttatgtacaacaattattatataaaacgacttgaacaattctgtgataaagaaaaaaaaaaaaaaaacgacttgaacaattcaacatgctatagatagtttatacgtaagctaataagcctgtttgatactgtatatgacatacgtgtagctgatatgatcaaactagacaaaattttctaatttctcccgtagatatatatttttagtttacttgggcattccatttctcatatgtttctgcaagttagcacacataatggtgacttggtgagttgctttttttctctctctttttcaatatgatatgctttcttcgatttttttattttttttaaacaatatatcatctaatcatggttttttcgtaatcgtggtttttctaattgtagaaatctagtctttttttttcgtttattttgtttttattagttcatcacaaataaacactgattggcaagaaggaaaaaaagaagatggttcgggagaagatgagaaaacacgagaaaacgcacaaaggttaacaaatagtaaaatagatagatagaaaacaaaacgagtcgagaaacttcgtatcaaatcttcaaccttcaagacctctccttttgtcacttagagccttctcatcctcacctacatatatacactcacttctacaattgtagtagatactcaacaaactcagaactagaattttctaatcttcactcagcgatatttttggctcatactcgaaacaa

SEQ ID NO: 9 - DNA, 659 bp, Hordeum vulgare
  source 1..659: /organism=“Hordeum vulgare” /mol_type=“unassigned DNA”
gtagagaattttaagatgtccatgaccacccgaccacccgctagctgcgtcattgtgtagtactctttctgtttttaaaataagtgtctgaactttatactaactttaatataaaattgtactaaacttgagacatttatttagacggagggagtagtatttatttacgtttaactattgtacttgctcgctgtaaactttaatgacagataacctctactgttaatcatgctattctggaaacggccccacaacccattcttgaatctagactagtcgcccccttgctggaattatacaattatttatagaaaaatctcacaaaaaaattattaacaaaaaaaaaagggagaaggcgccctaacggcaggttcgtgagtcccctggaagacgatagacccttcgagggttgggaacaggaaagaacgaatggctcacagcctacctggaaagcgaaagagaaacaaacactcgtggattggacagtcctcgagattcttcaagatcagtacctacgatgctataagtagtggctcaccttccatcgccctcccaactcaagccagccagcgtcaaaccaaagcgaacaacatcctacctacaatccgatacagattaatttcaacgcgagctacaaaccaaagcaagcaacaccgaca

SEQ ID NO: 10 - RNA, 135 nt, Hordeum vulgare
  source 1..135: /organism=“Hordeum vulgare” /mol_type=“unassigned RNA”
ugacagaagagagugagcacacagcggcaggcuacgucugauaagagcaugguuaauaguauagccaacugcuggcuauacuaugagcacgugcuuagacgcagcugcuguugcgugcucacccucucucugucu

SEQ ID NO: 11 - RNA, 21 nt, Hordeum vulgare
  source 1..21: /organism=“Hordeum vulgare” /mol_type=“unassigned RNA”
ugacagaagagagugagcaca

SEQ ID NO: 12 - DNA, 21 bp, Brassica napus
  source 1..21: /organism=“Brassica napus” /mol_type=“unassigned DNA”
ugacagaagagagugagcaca

SEQ ID NO: 13 - DNA, 24 bp, Brassica napus
  source 1..24: /organism=“Brassica napus” /note=“qRT-PCR pri-miRNA” /mol_type=“unassigned DNA”
gtcacatgcgtagagtgtgaaagg

SEQ ID NO: 14 - DNA, 24 bp, Brassica napus
  source 1..24: /organism=“Brassica napus” /note=“qRT-PCR pri-miRNA” /mol_type=“unassigned DNA”
ccatgtgtgctcactctcttctgt

SEQ ID NO: 15 - DNA, 23 bp, Arabidopsis thaliana
  source 1..23: /organism=“Arabidopsis thaliana” /note=“qRT-PCR pri-miRNA” /mol_type=“unassigned DNA”
tcctcccgagaagaagatgaaaa

SEQ ID NO: 16 - DNA, 23 bp, Arabidopsis thaliana
  source 1..23: /organism=“Arabidopsis thaliana” /note=“qRT-PCR pri-miRNA” /mol_type=“unassigned DNA”
gctaatcccaggttgtgctctct

SEQ ID NO: 17 - DNA, 49 bp, Arabidopsis thaliana
  source 1..49: /organism=“Arabidopsis thaliana” /note=“qRT-PCR” /mol_type=“unassigned DNA”
gtcgtatccagtgcagggtccgaggtattcgcactggatacgacgtgct

SEQ ID NO: 18 - DNA, 22 bp, Arabidopsis thaliana
  source 1..22: /organism=“Arabidopsis thaliana” /note=“qRT-PCR” /mol_type=“unassigned DNA”
tcgacgcgacagaagagagtga

SEQ ID NO: 19 - DNA, 50 bp, Arabidopsis thaliana
  source 1..50: /organism=“Arabidopsis thaliana” /note=“qRT-PCR” /mol_type=“unassigned DNA”
gtcgtatccagtgcagggtccgaggtattcgcactggatacgacgtgctc

SEQ ID NO: 20 - DNA, 22 bp, Arabidopsis thaliana
  source 1..22: /organism=“Arabidopsis thaliana” /note=“qRT-PCR” /mol_type=“unassigned DNA”
cgtcgacgtgacagaagaaaga

SEQ ID NO: 21 - DNA, 29 bp, Arabidopsis thaliana
  source 1..29: /organism=“Arabidopsis thaliana” /mol_type=“unassigned DNA”
ctcgagaagaaaaatggccatcccctagc

SEQ ID NO: 22 - DNA, 31 bp, Arabidopsis thaliana
  source 1..31: /organism=“Arabidopsis thaliana” /mol_type=“unassigned DNA”
ggatccgaggaattcactataaagagaatcg

SEQ ID NO: 23 - DNA, 42 bp, Arabidopsis thaliana
  source 1..42: /organism=“Arabidopsis thaliana” /mol_type=“unassigned DNA”
cttgacagaagataggagagagcattttctagagggagataa

SEQ ID NO: 24 - DNA, 42 bp, Arabidopsis thaliana
  source 1..42: /organism=“Arabidopsis thaliana” /mol_type=“unassigned DNA”
aaatgctctctcctatcttctgtcaagcttcggttcccctcg

SEQ ID NO: 25 - DNA, 29 bp, Arabidopsis thaliana
  source 1..29: /organism=“Arabidopsis thaliana” /note=“MIR156h” /mol_type=“unassigned DNA”
actcgagatagcgaagaggtaagaaagtg

SEQ ID NO: 26 - DNA, 30 bp, Arabidopsis thaliana
  source 1..30: /organism=“Arabidopsis thaliana” /note=“MIR156h” /mol_type=“unassigned DNA”
aggatccaaaacccaatttcaagattgatc

SEQ ID NO: 27 - DNA, 27 bp, Arabidopsis thaliana
  source 1..27: /organism=“Arabidopsis thaliana” /note=“qRT-PCR” /mol_type=“unassigned DNA”
ctgacagaagagagtgagcacacaaag

SEQ ID NO: 28 - DNA, 24 bp, Arabidopsis thaliana
  source 1..24: /organism=“Arabidopsis thaliana” /note=“qRT-PCR” /mol_type=“unassigned DNA”
cagatagagcagtgagcacgcaag

SEQ ID NO: 29 - DNA, 25 bp, Arabidopsis thaliana
  source 1..25: /organism=“Arabidopsis thaliana” /note=“qRT-PCR” /mol_type=“unassigned DNA”
cagaagagagtgagcacacaaaggg

SEQ ID NO: 30 - DNA, 26 bp, Arabidopsis thaliana
  source 1..26: /organism=“Arabidopsis thaliana” /note=“qRT-PCR” /mol_type=“unassigned DNA”
gtgagcacgcaaaagcaaccatatac